(19) United States
(12) Patent Application Publication
(10) Pub. No.: US 2008/0312738 A1
(43) Pub. Date: Dec. 18, 2008

(54) Multifocal Iol

(75) Inventor: Bernardus Franciscus Maria Wanders, Angerlo (NL)

Correspondence Address: SUGHRUE MION, PLLC, 2100 PENNSYLVANIA AVENUE, N.W., SUITE 800, WASHINGTON, DC, 20037, US

(73) Assignee: PROCORNEA HOLDINGS B.V., LZ Eerbeek (NL)

(21) Appl. No.: 11/993,139

(22) PCT Filed: Jun. 28, 2006
(86) PCT No.: PCT/NL06/50152

§ 371 Date: Aug. 4, 2008

(30) Foreign Application Priority Data
Jul. 1, 2005 (NL) 1029403

(51) Int. Cl. A61F 2/16

(52) U.S. Cl. 623/6.32

(57) ABSTRACT

Intraocular lens consisting of a centrally located lens part with supporting parts (haptics) on either side. According to the invention one supporting part is non-deformable and another supporting part, located approximately opposite said supporting part, is deformable. In this way it is achieved that when the ciliary cell contracts the reduction in the space therein is used for moving the centre of the lens part. By arranging the transition between distance and near part in a multifocal lens on said centre, switching from the distance part to the near part can be obtained for the user in an almost natural way.

### BACKGROUND OF THE INVENTION

The present invention relates to an intraocular lens (IOL) comprising a lens part with a centre, which lens part is provided on the periphery with two supporting parts (haptics). Lenses of this type are generally known in the state of the art. These are used for replacement of the eye lens after cataract operations, for example. For a large number of people an attempt is made to provide both a distance part and a near part (reading part) in these lenses. According to the state of the art this is obtained with the aid of mechanisms which act on the haptics and by which, when a lens system is used, the two or more lenses of such a system are moved in the axial direction. That is to say, the radial movement of the haptics is converted via a mechanism into an axial movement of the lenses. The focal length of the lens system can be changed by bringing two lenses linked one behind the other further apart or closer together and it is possible to provide a distance part and a near part. A lens system of this type is described in U.S. Pat. No 4,994,082.

However, it has been found that the movement of the ciliary muscle is relatively restricted, making it difficult to obtain the desired movement of the two or more lenses of the lens system. Moreover, it is not easy to implant a lens system of this type into the eye through a very small opening. This means that a larger opening has to be made with inevitable lengthening of the recovery time of the patient.

Therefore lenses of this type have not been generally introduced to date.

US-A1-2002/0019667 describes an IOL provided with various optical areas. The document describes an IOL which can be placed in the anterior chamber of the eye and a type which can be placed in the posterior chamber of the eye. All these types are permanently placed.

US-A1-2003/0018384 describes an IOL which can be implanted in the posterior chamber of the eye and is deformable along the optical axis, as a result of which the focal length of the IOL changes.

U.S. Pat. No. 4,955,902 describes an IOL for placing in an anterior chamber of an eye. The IOL has haptics of different length in order to hold the IOL in a fixed position in the eye eccentrically with respect to the pupil.

EP-A1-1.344.503 describes an IOL for the anterior chamber of the eye. The IOL is provided with haptics as engagement part for an insertion tool. The haptics additionally serve for gripping iris tissue.

### SUMMARY OF THE INVENTION

The aim of the present invention is to provide an IOL with which switching from one optical field to another optical field, such as a near part and a distance part, is possible in a simple way.

This aim is achieved with an IOL described above in that one supporting part is made radially non-deformable and in that another supporting part is made radially elastically deformable, such that when a radial compression force is applied to the IOL the centre of the lens part moves, the lens part being provided with two optical fields with different optical properties located near the centre.

According to the present invention the radial movement of the ciliary muscle is used for the radial movement of the centre of the lens with respect to the optical axis of the eye. By additionally arranging the separation between the different optical fields of the lens on or near the optical axis of the eye, shifting from, for example, the distance part to the near part will be obtained for the user almost automatically by the contraction of the ciliary muscle. A possible design of a lens with a distance part and a near part is described in PCT/NL96/00428 in the name of Procornea Holding B.V. The lens described there is a contact lens to be worn on the eye, but it has surprisingly been found that a lens of this type can also be used in an IOL. A lens of this type differs from other lenses in that the reading part is located within the (imaginary) boundary of the distance part. That is to say the reading part is on or within the radius of the outer boundary of the distance part (Rv). If a partial part is used this is preferably made as a segment which extends from the centre of the lens and has an aperture angle of between 90 and 180 degrees and more particularly of between 120 and 170 degrees.

The ratio between reading and distance part may mutually vary if two lenses are used. That is to say, one eye has a larger reading part than the other eye.

It has been found that a lens part of this type reacts particularly quickly to the relatively small radial movement of the ciliary muscle. This movement is between 0.1 and 1.5 mm. Moreover, in the above way the force generated by the ciliary muscle can be directly applied for moving the centre of the lens, that is to say no complicated lever mechanisms are needed to convert this movement into an axial movement. Consequently the force which has to be generated by the ciliary muscle for the elastic deformation of the supporting part involved can be relatively small. This force is in the range between 10 and 1000 mN.

In general an IOL of this type will be placed in the posterior chamber of the eye. With this arrangement the IOL will engage the ciliary muscle. In one embodiment the IOL has a diameter of approximately 11-13 mm, specifically approximately 11.5-12 mm. In this case the lens part usually has a diameter of approximately 6-8 mm, specifically 6-7 mm, although the trend at the moment is in the direction of this upper limit or even slightly larger.

In one embodiment the non-deformable supporting part is radially non-deformable at a maximum force to be exerted by a ciliary muscle. In practice this means that the radially non-deformable part does not deform at a compression force of less than about 100 mN. The non-deformable part often will not deform at a force of up to 1000 mN.

On the other hand the radially elastically deformable supporting part will have to deform radially when the ciliary muscle in an eye exerts a radially directed force thereon. The supporting part will in this case deform to a maximum extent at a maximum force to be exerted by said muscle. Furthermore, said supporting part will preferably hardly deform when the muscle does not exert any force thereon. The supporting part has, though, preferably been designed in such a way that when a force by the muscle is removed the supporting part causes the lens part to return to its initial position. In practice this means that the radially elastically deformable supporting part elastically deforms in the radial direction at a compression force of less than 1000 mN, in practice even at a compression force of less than about 200 mN. The supporting part will start to deform at a force of a minimum of 10 mN. Specifically the supporting part will deform at a force of between about 10 to 200 mN, in particular at a force of between about 10-100 mN.

The material used for the lens part and the haptics can be either hydrophilic or hydrophobic. For easy placing of the lens the various elements are preferably made so that they can be folded or rolled up. It will be understood that, if desired, the haptic can be made of a different material from the lens part in order to optimise the functional properties of the different parts.

Both the near part and the distance part can be made both with single strength and as multifocal. The reading part is preferably at the bottom of the lens because this corresponds to the natural inclination of people to look down when reading.

Apart from in the corrective distance part and near part described above, further corrections can be made in the lens part to correct particular optical abnormalities.

According to a further advantageous embodiment of the invention there are two lens parts placed one behind the other in the axial direction. One lens part is made as described above. The other lens part is likewise a lens part provided with one non-deformable haptic and one deformable haptic. However, they are fitted in precisely the reverse positions. This means that when the first lens part moves upwards when the ciliary muscle contracts the second lens part will move downwards with respect to the optical axis of the eye. An additional enhancing effect can be obtained by the combined action of the two lens parts. The second additional lens part is preferably made as a prism or other image compensator, wherein the part with the smallest dimension of the prism is located near the deformable haptic.

All the lens parts described above can be provided with a marking, whereby it is possible to assess and if necessary correct the position of the lens part with respect to the eye. This is important for implantation and possible correction.

The invention further relates to an intraocular lens (IOL) for implantation in a posterior chamber of the eye, wherein the IOL has a diameter of approximately 11-13 mm and comprises a lens part with a centre, which lens part is provided on the periphery with two supporting parts (haptics), the supporting parts being made mutually differently radially deformable under the influence of a radial compression force, with a mutual difference such that when a radial compression force is applied to the IOL the centre of the lens part moves, the lens part being provided with two optical fields with different optical properties located near the centre.

The invention further relates to an intraocular lens (IOL) for implantation in a posterior chamber of the eye, wherein the IOL has a diameter of approximately 11-13 mm and comprises a first lens part with a centre, which first lens part is provided on the periphery with two supporting parts (haptics), wherein the IOL comprises a second lens part axially behind the first lens part, interconnected by means of the supporting parts, which supporting parts are made mutually differently radially deformable under the influence of a radial compression force, with a mutual difference such that when a radial compression force is applied to the IOL, the centre of the first and second lens parts move mutually radially.

The invention furthermore relates to an intraocular lens (IOL) for implantation in a posterior chamber of the eye, wherein the IOL has a diameter of approximately 11-13 mm and comprises a first lens part with a centre, which first lens part is provided on the periphery with two supporting parts (haptics), wherein the IOL comprises a second lens part axially behind the first lens part, wherein the second lens part is connected to the first lens part via supporting parts that are connected to the supporting parts, wherein one supporting part is non-deformable in the radial direction and a additional supporting part is radially deformable such that when a radial compression force is applied to the IOL, the centre of the first and second lens parts move mutually radially.

The invention further relates to the use of an intraocular lens as described in this document for implantation in a posterior chamber of the eye to replace an eye lens.

The intraocular lens described above can be made in any way known in the state of the art. In addition, it is possible to make the lens part and the haptic separately and to connect them together later. However, it is also possible to make them as one entity. According to an advantageous embodiment they are made as one entity by (injection) moulding. The subsequent processing is turning. As described in PCT/NL96/00428, during such a turning operation the bit can be moved every revolution to and fro in the direction parallel to the rotational axis. This makes it possible to produce the lens part by turning. It is also possible according to an advantageous embodiment to perform the turning so finely that a subsequent polishing operation can be omitted. The material of the lens can be any desired material.

### SHORT DESCRIPTION OF THE DRAWINGS

The invention will be explained in more detail below with reference to illustrative embodiments depicted in the drawings, where

FIGS. 1a, b show in front view and side view a first embodiment of the present invention in the case of a non-contracted ciliary muscle;

FIGS. 2a, b show the embodiment according to FIG. 1 with a contracted ciliary muscle; and

FIG. 3 shows a further embodiment of the present invention wherein the IOL is made up of a lens system.

### DESCRIPTION OF EMBODIMENTS

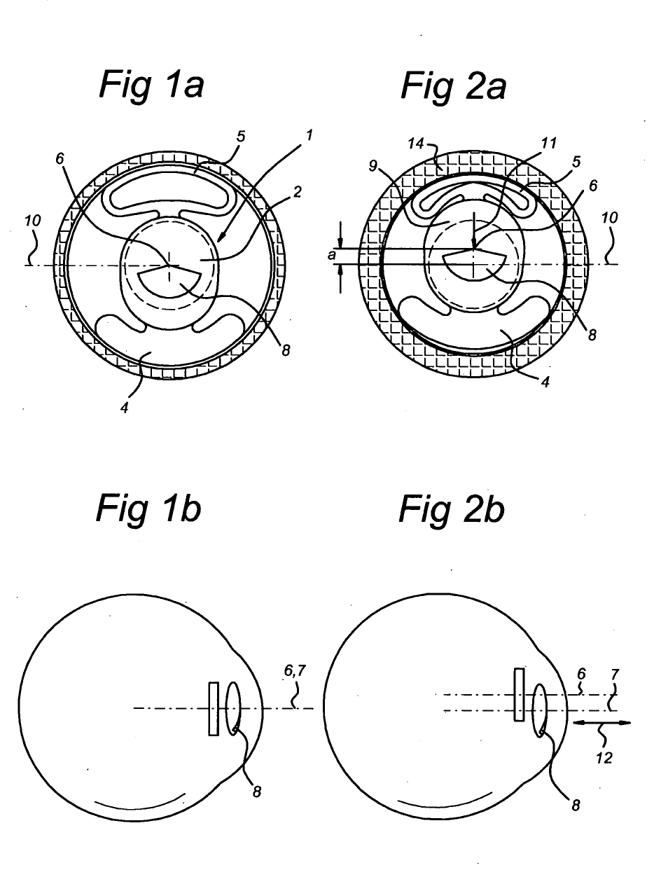

In FIG. 1 and more particularly FIG. 1a an intraocular lens is indicated in its entirety by 1. This consists of a lens part 2 where so-called haptics or supporting parts 4 and 5 are provided on opposite sides. In the present case two supporting parts located opposite one another are illustrated. It should be understood that, instead of two, there may be three or more supporting parts.

In the present illustrative embodiment the supporting part 5 has been made elastically deformable, while the supporting part 4 is rigid. The lens part 2 has an optical centre 6 and a horizontal longitudinal centre line 10. A distance part 9 and a reading part 8 are delimited in the lens part. The reading part 8 extends between 120 and 160 degrees.

The distance of the supporting parts 4 and 5 from the outer periphery is made such that when the haptic is not contracted the optical axis 7 of the eye coincides with the optical centre 6 of the lens. This is illustrated in FIG. 1b. Consequently the user can use the lens for both near vision and distance vision with little effort.

The rigid supporting part 4 is in this case substantially solid, while the deformable supporting part 5 is hollow.

The supporting part 5 has in this case, for example, a bearing surface that is in contact with, or engages, the ciliary muscle. Said bearing surface is connected by means of elastic connecting parts to the lens part 2. Alternatively the bearing surface can be deformable and the connecting parts rigid, or both can be elastically deformable. It is also conceivable to use one connecting part.

FIG. 2 shows the state during contracting of the ciliary muscle 14. Because the non-deformable supporting part 4 is not deformable, all movement will need to come from the elastically deformable supporting part 5. This means that when the ciliary muscle contracts the centre 6 of the lens part 2 is moved upwards with respect to the optical axis 7. Such a movement is indicated in FIG. 2a by a, where a can be between 0.1 and 1.5 mm.

The radial direction is indicated by arrow 11, while arrow 12 in FIG. 2b indicates the axial direction.

By constriction of the ciliary muscle 14, which normally takes place when starting to read anyway, the reading part 8 will be pushed in front of the user's optical axis. If the user at the same time looks downwards, as is usual, reading can be optimised.

It should be understood that a further structure, which makes it possible to correct all kinds of optical abnormalities, such as astigmatism, can be arranged at the back of the lens.

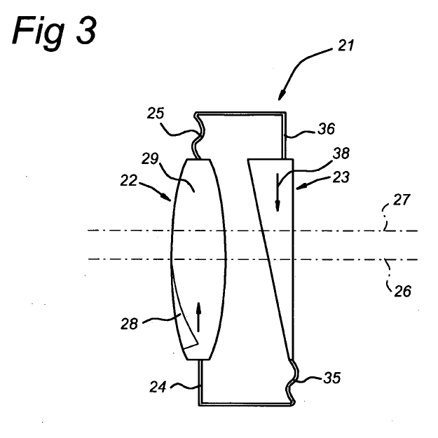

FIG. 3 illustrates a variant of the invention. The intraocular lens is indicated there in its entirety by 21 and consists of a lens part 22 and an additional lens part 23. The lens part 22 is arranged inside the ciliary muscle in the way described above, there being a deformable supporting part or haptic 25 and a non-deformable supporting part or haptic 24. The distance part is indicated by 29 and the near part by 28. The centre of the lens is 26, while the optical axis of the eye concerned is indicated by 27.

The additional lens part 23 is made as a prism that tapers in a downward direction.

Opposite the non-deformable supporting part 23 is a deformable additional supporting part 35 of the additional lens 23. A non-deformable additional supporting part of the additional lens 23 is indicated by 36 and is located at the height of the deformable supporting part 25.

When the ciliary muscle contracts, lens 22 will be displaced upwards with respect to the optical axis of the eye concerned. The prism will perform an exactly opposite movement, in other words downwards as indicated by arrow 38. Because of this the effect of the image shift from the distance part to the reading part can be compensated or provision for further corrections in the eye can be made. Here too it is the case that the radial contraction of the ciliary muscle in the direction of arrow 11 (FIG. 2) is directly converted into a radial movement of the respective lens parts 22 and 23 concerned.

It will be understood that the reading part could also be located at the top of the lens part. It will also be understood that other corrections are possible with the present invention.

After reading the above description variants that fall within the scope of are obvious and/or fall within the scope of the appended claims will be immediately apparent to those skilled in the state of the art. Moreover, it should be understood that the measures described in the subordinate claims can also be taken separately without combination with the independent claims.

1. Intraocular lens (IOL) comprising a lens part with a centre, which lens part is provided on the periphery with two supporting parts (haptics), wherein one supporting part is made radially non-deformable and wherein another supporting part is made radially elastically deformable, such that when a radial compression force is applied to the IOL the centre of the lens part moves, the lens part being provided with two optical fields with different optical properties located near the centre.

2. Intraocular lens, wherein the supporting part located at the top in use is made radially elastically deformable and the part located at the bottom is made radially non-deformable.

3. Intraocular lens according to claim 1, wherein in use an optical field is located above the horizontal longitudinal centre line through the centre of the lens part and an optical field is located below the horizontal longitudinal centre line.

4. Intraocular lens according to claim 3, wherein said field located above the horizontal longitudinal centre line is a distance part and the part located below the horizontal longitudinal centre line is a near part.

5. Intraocular lens according to claim 3, wherein starting from a distance part extending over the entire surface area of said lens part, said near part is delimited within the thickness of said (imaginary) distance part.

6. Intraocular lens according to claim 1, comprising an additional lens part placed axially in front of or behind said lens part and provided with an additional non-deformable supporting part and an additional deformable supporting part, wherein said additional non-deformable supporting part is located axially in front of or behind said deformable supporting part and said additional deformable supporting part is located axially in front of or behind said non-deformable supporting part.

7. Intraocular lens according to claim 6, wherein said additional lens part comprises an image compensator.

8. Intraocular lens according to claim 7, wherein said image compensator has the smallest dimension near the additional deformable supporting part.

9. Intraocular lens according to claim 1, wherein the non-deformable supporting part does not radially deform when there is a radial compression force on said supporting part of up to 100 mN, specifically up to 1000 mN.

10. Intraocular lens according to claim 1, wherein the deformable supporting part radially deforms when there is a radial compression force on said supporting part of less than about 1000 mN, specifically the supporting part deforms at a force of a minimum of about 10 mN.

11. Intraocular lens according to claim 1, adapted for implantation in the posterior chamber of the eye.

12. Intraocular lens according to claim 1, wherein the IOL has a diameter of 11-13 mm, in particular in the range of approximately 11.5-12 mm.

13. Intraocular lens (IOL) for implantation in a posterior chamber of the eye, wherein the IOL has a diameter of approximately 11-13 mm and comprises a lens part with a centre, which lens part is provided on the periphery with two supporting parts (haptics), characterised in that the supporting parts are made mutually differently radially deformable under the influence of a radial compression force, with a mutual difference such that when a radial compression force is applied to the IOL the centre of the lens part moves, the lens part being provided with two optical fields with different optical properties located near the centre.

14. Intraocular lens (IOL) for implantation in a posterior chamber of the eye, wherein the IOL has a diameter of approximately 11-13 mm and comprises a first lens part with a centre, which first lens part is provided on the periphery with two supporting parts (haptics), wherein the IOL comprises a second lens part axially behind the first lens part, interconnected by means of the supporting parts, which supporting parts are made mutually differently radially deformable under the influence of a radial compression force, with a mutual difference such that when a radial compression force is applied to the IOL, the centre of the first and second lens parts move mutually radially.

15. Intraocular lens (IOL) for implantation in a posterior chamber of the eye, wherein the IOL has a diameter of approximately 11-13 mm and comprises a first lens part with a centre, which first lens part is provided on the periphery with two supporting parts (haptics), wherein the IOL comprises a second lens part axially behind the first lens part, and wherein the second lens part is connected to the first lens part via supporting parts that are connected to the supporting parts, wherein one supporting part is non-deformable in the radial direction and an additional supporting part is radially deformable such that when a radial compression force is applied to the IOL, the centre of the first and second lens parts move mutually radially.

16. Use of an intraocular lens according to claim 1 for implantation in a posterior chamber of the eye to replace an eye lens.

17. Use of an intraocular lens according to claim 13 for implantation in a posterior chamber of the eye to replace an eye lens.

18. Use of an intraocular lens according to claim 14 for implantation in a posterior chamber of the eye to replace an eye lens.

19. Use of an intraocular lens according to claim 15 for implantation in a posterior chamber of the eye to replace an eye lens.
